(19) United States
(12) Patent Application Publication
(10) Pub. No.: US 2022/0339326 A1
(43) Pub. Date: Oct. 27, 2022

(54) REUSABLE URINARY CATHETER PRODUCTS

(71) Applicant: Hollister Incorporated, Libertyville, IL (US)

(72) Inventors: David J. Farrell, Ballina (IE); Padraig M. O'Flynn, Ballina (IE); John T. Clarke, Galway (IE)

(21) Appl. No.: 17/761,799

(22) PCT Filed: Sep. 22, 2020
(86) PCT No.: PCT/US2020/052051

§ 371 Date: Mar. 18, 2022

Related U.S. Application Data
(60) Provisional application No. 62/905,056, filed on Sep. 24, 2019.

(51) Int. Cl. A61L 29/12; A61L 2/10; A61L 29/16

(52) U.S. Cl. CPC A61L 29/126; A61L 2/10; A61L 29/16; A61L 2202/122; A61L 2300/102; A61L 2400/10; A61L 2420/04

(57) ABSTRACT

Reusable intermittent catheter products comprising a hygienic catheter including photoactive titanium dioxide.

The present application claims the benefit of and priority to U.S. Provisional Application No. 62/905,056, filed Sep. 24, 2019, which is hereby incorporated herein by reference.

### FIELD OF THE DISCLOSURE

The present disclosure generally relates to urinary catheters. More particularly, the present disclosure relates to reusable urinary catheter products.

### BACKGROUND

Catheters are used to treat many different types of medical conditions and typically include an elongated shaft that is inserted into and through a passageway or lumen of the body. Catheters, and in particular intermittent catheters, are commonly used by those who suffer from various abnormalities of the urinary system, such as urinary incontinence. With the advent of intermittent catheters, individuals with urinary system abnormalities can self-insert and self-remove intermittent catheters several times a day.

Urinary catheters are frequently provided as disposable, single-use items. A user will remove the catheter from a package, use the catheter once, and then dispose of the catheter and the package. Reusable urinary catheters could, thus, be advantageous in reducing the amount of waste created by the use of disposable catheters, but there are various challenges associated with the use of reusable catheters (including storage, transport, and sterilization) that must be overcome before widespread acceptance and use of reusable catheters.

There is a need for reusable catheter products and methods of sterilizing the same.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a reusable urinary catheter product is disclosed. The product includes an intermittent catheter comprising a catheter shaft, wherein the catheter shaft comprises polymer and photoactive titanium dioxide.

In another aspect, a method of disinfecting a reusable catheter comprising photoactive titanium dioxide is disclosed. The method includes exposing the catheter to light to activate the photoactive titanium dioxide.

### BRIEF DESCRIPTION OF THE DRAWINGS

FIG. 1 is perspective view of a reusable catheter in accordance with the present disclosure;

FIG. 2 is a partial enlarged cross-sectional view of one embodiment of a catheter shaft of FIG. 1;

FIG. 3 is a partial enlarged cross-sectional view of another embodiment of a catheter shaft of FIG. 1;

FIG. 4 is a schematic illustration of a catheter product including a light source; and

FIG. 5 is a front elevational view of another embodiment of a reusable catheter product including a case.

### DESCRIPTION

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

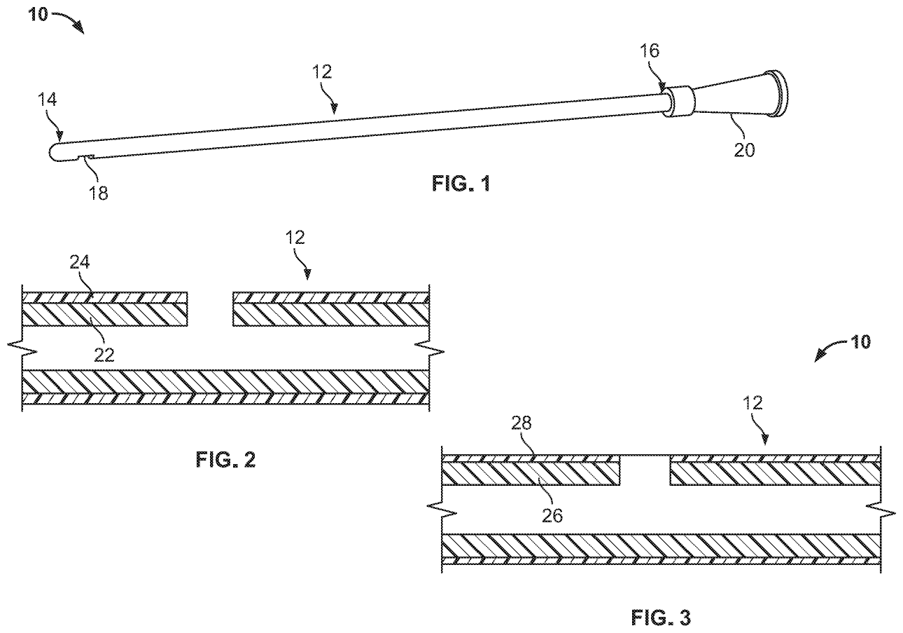

Reusable urinary catheter products according to the present disclosure and their individual components may be variously configured without departing from the scope of the present disclosure, but in one embodiment, a reusable urinary catheter 10 is configured as shown in FIG. 1.

FIG. 1 illustrates one embodiment of a reusable intermittent catheter 10 of the present disclosure. The intermittent catheter 10 may be used, disinfected and then reused. The reusable catheter 10 includes a catheter shaft 12. The catheter shaft 12 includes a proximal insertion end 14 and a distal end 16. The proximal insertion end 14 includes a drainage hole or eyelet 18 for the drainage of urine from the bladder and into the catheter shaft 12. A drainage member 20, such as a funnel, is located at the distal end 16 of the catheter shaft 12.

The catheter shaft 12 is made from polymer(s) and includes photoactive titanium dioxide. Furthermore, the drainage member 20 also may be made from polymer(s) and include a photoactive titanium dioxide. The polymer(s) of the shaft and the drainage member may be the same or different polymers. Such polymers may include thermoplastic polymers, such as polyvinyl chloride, thermoplastic olefins, such as polypropylene, polyethylene, block copolymer polypropylene, and/or thermoplastic elastomers, such as Styrenic block copolymers, polyurethanes etc. The polymers may include biodegradable polymers and biodegradable thermoplastic polymers.

Photoactive titanium dioxide, when activated with light, provides a disinfectant that kills and/or inhibits the growth of microbes on the catheter surface. When photoactive titanium dioxide absorbs light with suitable photon energies, the titanium dioxide reacts with oxygen from the surrounding environment (air and/or, if present water). This reaction provides a self-cleaning and/or self-disinfecting effect. When a catheter includes photoactive titanium dioxide, the catheter will include self-cleaning and/or self-disinfecting characteristics.

Several organic molecules and microorganisms are destroyed or killed by oxidation. The antibacterial properties of a light activated titanium dioxide surface can be derived from the combination of two different characteristics of photoactive titanium dioxide containing materials, namely, self-cleaning and self-disinfection characteristics. The self-cleaning characteristic provides anti-bacterial growth and/or anti-bacterial adhesion characteristics to the catheter surface. For example, degradation of organic substances by oxidation prevents bacteria and biofilm adhesion on the surface of the catheter. Furthermore, degradation of the organic material may kill or destroy the bacteria, thereby providing disinfection. When a surface containing titanium dioxide absorbs light (such as UV light, near UV light and/or visible light) the titanium dioxide reacts with surrounding oxygen to form hydroxyl (OH) radicals. Hydroxyl free radicals are very powerful oxidizing agents that attack the structure of cell membranes and break down organic molecules. Thus, providing a self-cleaning and/or self-disinfecting catheter surface.

The titanium dioxide may be anatase, rutile, brookite or mixtures thereof. In solids, the outer electrons can potentially be found in two energetic bands, namely, the valence band and the conduction band. The energy gap between the valence band and the conduction band for titanium dioxide is in the region of 3.0-3.5 eV. With sufficient energy from exposure to light, electrons in titanium oxide can move from the valence band to the conduction band, thus initiating or activating a reaction with the titanium dioxide and surrounding oxygen.

Anatase shows a band gap of 3.2 e V, corresponding to a UV wavelength adsorption of 385 nm. In contrast, rutile has a smaller band gap of 3.0 eV, with excitation wavelengths that extend into the visible light range of 410 nm. In one embodiment, the catheters of the present disclosure may include a mixture of the different species of photoactive titanium dioxide. For example, the titanium dioxide could be a mixture of anatase-rutile or could be a mixture of brookite-anatase. In some instances the mixtures could be more active than the species is alone. In one embodiment, the photoactive titanium dioxide includes a mixture of anatase in an amount between 60% and 90% and rutile in an amount between 10% and 40%. In another alternative, the photoactive titanium dioxide includes a mixture of 80% anatase and 20% rutile. Furthermore, the catheter could also include one or more of zinc oxide, silver oxide and silicon dioxide. The catheter could include other additives as well. Additionally, the photoactive titanium dioxide may be in microparticle or nanoparticle form.

Photoactive titanium dioxide may be incorporated into the catheter and/or catheter surface in any suitable manner. For example, a portion of the catheter, such as the shaft and/or drainage member, may be made from a polymer that has been compounded (mixed or blended) with photoactive titanium dioxide. Polymer pellets may be mixed and melted with photoactive titanium dioxide to form a compounded polymer. The compounded polymer may then be extruded or molded to form the catheter or portions thereof.

Referring to FIG. 2, the catheter shaft may be made by coextrusion. The coextruded catheter shaft may have an inner layer 22 and an outer layer 24. One or both of the inner layer 22 and the outer layer 24 may be made from a polymer compounded with photoactive titanium dioxide. In one embodiment, the outer layer 24 is made from a polymer compounded with photoactive titanium dioxide, while the inner layer 22 is made from a polymer with or without other additives.

Referring to FIG. 3, in this embodiment, the catheter 10 includes a substrate polymer that defines, for example, the catheter shaft 12 and/or the drainage member 20. In the embodiment illustrated in FIG. 3, a cross-section of the catheter shaft 12 is shown for illustrative purposes. The catheter shaft 12 includes a substrate 26 formed from a polymer. The polymer may be extruded or molded to form the substrate 26. Disposed over the surface of the substrate 26 is a coating 28 including photoactive titanium dioxide.

In one embodiment, the coating includes a lubricious hydrophilic polymer, wherein the photoactive titanium dioxide is contained in the hydrophilic coating. The lubricious hydrophilic polymer may be a cross-linked polymer. Furthermore, the lubricious hydrophilic polymer may include a matrix, wherein the photoactive titanium dioxide is contained within the matrix. The hydrophilic polymer may be any suitable hydrophilic polymer, such polyvinylpyrrolidone, polyethylene oxide, polyacrylic acid, polyacrylamide, hyaluronic acid, polyvinyl alcohol etc.

Alternatively, the photoactive titanium oxide may be applied to the surface of the substrate 26 by vapor deposition coating. In one embodiment, the coating is applied by chemical vapor deposition.

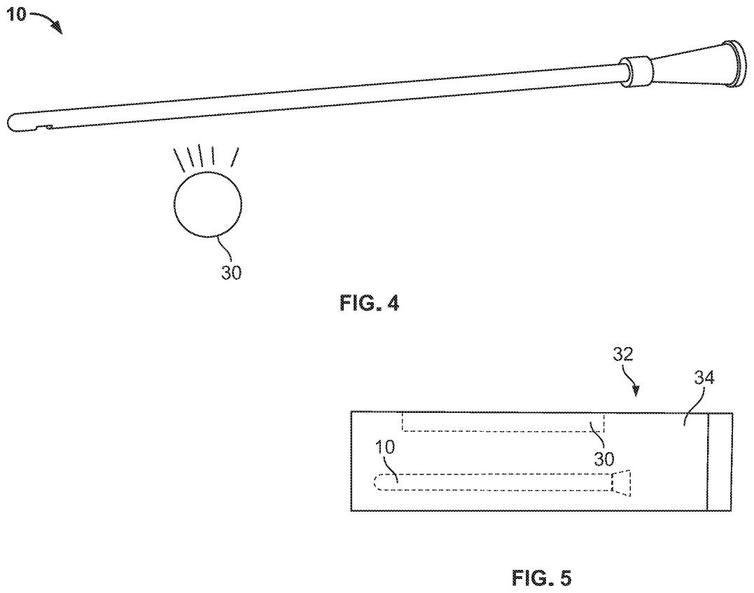

Referring now to FIG. 4, any of the reusable catheter products disclosed herein may include a light source 30 for exposing the catheter 10 to light. Depending on the particular application, the light source 30 may produce UV light, near UV light or visible light for activating the titanium dioxide. The light source may be, for example, one or more light emitting diodes. Furthermore, the light source may be powered by batteries, which may be rechargeable batteries. As discussed above, when the catheter is exposed to light, the photoactive titanium dioxide is activated to react with oxygen in the surrounding environment. The oxygen may be gas from the surrounding air or may be oxygen from a surrounding liquid, such as water. For example, when the catheter includes a lubricious hydrophilic coating, the hydrophilic coating is activated to be lubricious by hydration with a hydration fluid, such as water. The hydration fluid may be present at the same time that the catheter is exposed to light. The light source 30 may be associated with an automatic shutoff switch or timer that shuts off the light after a selected time.

Referring to FIG. 5, there is shown one embodiment of a reusable catheter product 32. The product 32 includes a case 34 holding a catheter 10 and a light source 30. The catheter 10 may be any of the photoactive titanium dioxide catheters disclosed herein, and the light source 30 may be any of the light sources disclosed herein. In use, the user removes the catheter 10 from the case 34, and performs catheterization. The user then puts the catheter 10 back in the case 34 and activates the light source 30 to expose the catheter 10 to light, which activates the photoactive titanium dioxide. After the exposure to the light source, the catheter is ready for use. When the catheter 10 includes a hydrophilic coating, the case may also include a hydration fluid for hydrating the hydrophilic coating. The case may also include an indicator that indicates that the catheter has been exposed to a sufficient level of light or light energy. For example, the case may include a UV indication strip within the case that changes color upon exposure to selected amount of UV light. Such strips may be tuned to change color when exposed to a certain level of UV radiation.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A reusable urinary catheter product, comprising:
an intermittent catheter comprising a catheter shaft, wherein the catheter shaft comprises polymer and photoactive titanium dioxide.

2. The product of claim 1, wherein the catheter further includes a drainage member comprised of polymer and photoactive titanium dioxide.

3. The product of claim 1, wherein the polymer is compounded with photoactive titanium dioxide.

4. The product of claim 3, wherein the catheter shaft includes a coextruded tube including an inner polymer layer and an outer polymer layer, wherein the outer polymer layer comprises the polymer compounded with photoactive titanium dioxide.

5. The product of claim 1, wherein the polymer defines a substrate and a coating comprising the photoactive titanium dioxide is disposed on a surface of substrate.

6. The product of claim 5, wherein in the coating comprises a lubricious hydrophilic polymer and the photoactive titanium dioxide is contained in the hydrophilic coating.

7. The product of claim 6, wherein the lubricious hydrophilic polymer is cross-linked.

8. The product of claim 6, wherein the lubricious hydrophilic polymer comprises a matrix and the photoactive titanium dioxide is contained within the matrix.

9. The product of claim 5, wherein the coating is a vapor deposition coating.

10. The product of claim 5, wherein the coating is a chemical vapor deposition coating.

11. The product of claim 1, further including a light source that produces light that activates the photoactive titanium dioxide.

12. The product of claim 11, wherein the light source produces UV-light, near UV-light, visible light and/or mixtures thereof.

13. The product of claim 11, further an automatic shut off switch that shuts off the light source after a selected period of time.

14. The product of claim 11, further including a storage and disinfectant case containing the light source and the intermittent urinary catheter.

15. The product of claim 1, wherein the photoactive titanium dioxide comprises anatase, rutile, brookite, or mixtures thereof.

16. The product of claim 1, wherein the photoactive titanium dioxide comprises a mixture of anatase in an amount between 90% and 60% and rutile in an amount between 10% and 40%.

17. The product of claim 1, wherein the photoactive titanium dioxide comprises a mixture of 80% anatase and 20% rutile.

18. The product of claim 1, wherein the catheter shaft further comprises one of more of zinc oxide, silver oxide and silicon dioxide.

19. A method of disinfecting a reusable catheter comprising photoactive titanium dioxide, comprising:
exposing the catheter to light to active the photoactive titanium dioxide.

20. The method of claim 19, wherein the catheter comprises polymer that is compounded with photoactive titanium dioxide.

21-33. (canceled)
